(19) United States
(12) Patent Application Publication
(10) Pub. No.: US 2024/0390653 A1
(43) Pub. Date: Nov. 28, 2024

(54) CATHETER SYSTEM HAVING A GUIDEWIRE AND A NEEDLE SAFETY CLIP

(71) Applicant: Becton, Dickinson and Company, Franklin Lakes, NJ (US)

(72) Inventor: Clark Ragsdale, Herriman, UT (US)

(21) Appl. No.: 18/658,795

(22) Filed: May 8, 2024

Related U.S. Application Data
(60) Provisional application No. 63/468,946, filed on May 25, 2023.

(51) Int. Cl. A61M 25/06; A61M 25/00; A61M 25/09

(52) U.S. Cl. CPC A61M 25/0612; A61M 25/0097; A61M 25/09

(57) ABSTRACT

A catheter system may include a catheter assembly and a needle safety clip disposed within a safety can proximal to the catheter assembly. The catheter system may include a barrel, a needle hub fixed to the barrel, an introducer needle secured within the needle hub, a catheter hub slider, a guidewire slider, and a guidewire coupled to the guidewire slider. In response to the movement of the catheter system from a first position to a second position, the guidewire slider may push out on a flexible arm of catheter hub slider such that the flexible arm is removed from contact with the needle hub, and the catheter hub slider is configured to slide from a catheter hub slider proximal position to a distally-advanced position.

### RELATED APPLICATIONS

This application claims the benefit of U.S. Provisional Patent Application No. 63/468,946, which was filed on May 25, 2023, which is incorporated herein in its entirety.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices, systems, and methods. In particular, the present disclosure relates to a catheter system having a guidewire and needle safety clip, as well as related devices and methods.

In some embodiments, the catheter system may include a catheter assembly, which may include a catheter hub. In some embodiments, the catheter hub may include a distal end, a proximal end, and a lumen extending through the distal end of the catheter hub and the proximal end of the catheter hub. In some embodiments, the catheter system may include a catheter, which may include a distal tip and which may extend from the distal end of the catheter hub. In some embodiments, the catheter system may include a needle safety clip disposed within a safety can disposed proximal to the catheter hub.

In some embodiments, the catheter system may include a barrel, which may include a distal end and a proximal end. In some embodiments, the catheter system may include a needle hub fixed to the barrel. In some embodiments, the catheter system may include an introducer needle, which may include a sharp distal tip and a proximal end. In some embodiments, the proximal end of the introducer needle may be secured within the needle hub.

In some embodiments, the catheter system may include a catheter hub slider, which may include a flexible arm. In some embodiments, the flexible arm may be within and part of a distally-extending arm of the catheter hub slider. In some embodiments, the catheter system may include a guidewire, which may include a first end and a second end. In some embodiments, the first end of the guidewire may be configured to advance distally through the introducer needle. In some embodiments, the catheter system may include a guidewire slider.

In some embodiments, the catheter system may be configured to move from a first position to a second position. In some embodiments, when the catheter system is in the first position, the guidewire slider may be disposed in the guidewire slider proximal position and configured to slide from a guidewire slider proximal position to a distal position to distally advance the guidewire. In some embodiments, when the catheter system is in the first position, the catheter hub slider may be disposed in a catheter hub slider proximal position. In some embodiments, when the catheter system is in the first position, the flexible arm of the catheter hub slider may contact the needle hub to prevent the catheter hub slider from sliding from a catheter hub slider proximal position to a distally-advanced position.

In some embodiments, when the catheter system is in the second position, the guidewire slider may be disposed in the distal position such that the guidewire is advanced beyond the distal tip of the catheter. In some embodiments, when the catheter system is in the second position, the catheter hub slider may be disposed in the catheter hub slider proximal position. In some embodiments, when the catheter system is in the second position, the distally-extending arm of the catheter hub slider may be coupled to the catheter assembly to prevent distal movement and proximal movement of the catheter hub slider with respect to the catheter hub. In some embodiments, in response to the movement of the catheter system from the first position to the second position, the guidewire slider may push out on the flexible arm such that the flexible arm is removed from contact with the needle hub, and the catheter hub slider is configured to slide from a catheter hub slider proximal position to a distally-advanced position. In some embodiments, when the catheter system is in the second position, the guidewire slider may be configured to slide back and forth between the guidewire slider proximal position and the distal position.

In some embodiments, the second end of the guidewire may be fixed within the barrel. In some embodiments, the second end of the guidewire may be fixed within the needle hub. In some embodiments, the guidewire slider may include a U-shaped slot therethrough. In some embodiments, when the catheter system is in the first position, the guidewire may be disposed in the U-shaped slot in a double-throw configuration such that there is a 2:1 advancement ratio between the first end of the guidewire and the guidewire slider. In some embodiments, the U-shaped slot is oriented vertically within the barrel. In some embodiments, the U-shaped slot may be asymmetric.

In some embodiments, the catheter may be configured to move from the second position to a third position. In some embodiments, when the catheter system is in the third position, the guidewire slider is disposed in the distal position such that the guidewire is advanced beyond the distal tip of the catheter. In some embodiments, when the catheter system is in the third position, the catheter hub slider may be disposed in the distally-advanced position, and distal advancement of the catheter hub slider advances the catheter assembly.

In some embodiments, the distally-advanced position of the catheter hub slider is a partially distally-advanced position. In some embodiments, the safety can may include a groove. In some embodiments, the barrel may include an inner ramp. In some embodiments, the catheter system may be configured to move from the third position to a fourth position. In some embodiments, when the catheter system is in the fourth position, the guidewire slider is disposed in the distal position such that the guidewire is advanced beyond the distal tip of the catheter. In some embodiments, when the catheter system is in the fourth position, the catheter hub slider may be disposed in a fully distally-advanced position.

In some embodiments, in response to movement of the catheter system from the third position to the fourth position, the distally-extending arm of the catheter hub slider may contact the inner ramp on the inner surface of the barrel to remove the distally-extending arm from the groove of the safety can such that the catheter hub slider is uncoupled from the catheter assembly. In some embodiments, the distally-extending arm may include a hook and an outwardly-extending protrusion. In some embodiments, in response to movement of the catheter system from the third position to the fourth position, the outwardly-extending protrusion of the catheter hub slider may contact the inner ramp on the inner surface of the barrel to remove the hook from the groove of the safety can such that the catheter hub slider is uncoupled from the catheter assembly.

In some embodiments, the distally-extending arm may be a first distally-extending arm. In some embodiments, the catheter hub slider may include a second distally-extending arm opposite the first distally-extending arm. In some embodiments, in response to movement of the catheter system from the third position to the fourth position, the second distally-extending arm of the catheter hub slider may contact the inner ramp on the inner surface of the barrel to remove the second distally-extending arm from the groove of the safety can such that the catheter hub slider is uncoupled from the catheter assembly.

In some embodiments, when the catheter system is in the fourth position, the barrel may be moved proximally with respect to the catheter assembly to withdraw the introducer needle from the catheter assembly. In some embodiments, in response to proximal withdrawal of the introducer needle past the needle safety clip, the needle safety clip may uncouple from the catheter hub. In some embodiments, when the catheter system is in the first position, the second position, the third position, and the fourth position, the introducer needle may extend through the catheter. In some embodiments, the flexible arm may include a fin tapered inwardly in a distal direction. In some embodiments, in response to the movement of the catheter system from the first position to the second position, the guidewire slider may push out on the fin such that the flexible arm is removed from contact with the needle hub, and the catheter hub slider is configured to slide from the catheter hub slider proximal position to the distally-advanced position.

In some embodiments, the flexible arm may be a first flexible arm. In some embodiments, the catheter hub slider may include a second flexible arm opposite the first flexible arm. In some embodiments, the second flexible arm of the catheter hub slider may contact the needle hub to prevent the catheter hub slider from sliding from the catheter hub slider proximal position to the distally-advanced position. In some embodiments, in response to the movement of the catheter system from the first position to the second position, the guidewire slider may push out on the second flexible arm such that the second flexible arm is removed from contact with the needle hub, and the catheter hub slider is configured to slide from a catheter hub slider proximal position to a distally-advanced position. In some embodiments, the catheter assembly may include another safety clip disposed within the safety can and configured to trap the sharp distal tip of the introducer needle in response to proximal withdrawal of the introducer needle.

In some embodiments, the barrel may include a slot disposed between the distal end of the barrel and the proximal end of the barrel. In some embodiments, when the catheter system is in the first position, the guidewire slider may extend through the slot and may be configured to slide along the slot from the guidewire slider proximal position to the distal position to distally advance the guidewire. In some embodiments, when the catheter system is in the second position, the guidewire slider may extend through the slot and may be configured to slide along the slot from the guidewire slider distal position to the catheter hub proximal position.

In some embodiments, the proximal end of the barrel may include an opening. In some embodiments, the catheter hub slider may be configured to slide within the opening from the proximal catheter hub slider position to the distally-advanced position. In some embodiments, the guidewire slider may be configured to slide within the opening from the guidewire slider proximal position to the distal position. In some embodiments, the guidewire slider may wrap around the catheter hub slider. In some embodiments, the guidewire slider may wrap completely around the catheter hub slider.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of the catheter system in an example first position, according to some embodiments;

FIG. 1B is a partial cutaway view of the catheter system in the first position, according to some embodiments;

FIG. 1C is an upper perspective view of an example catheter hub slider of the catheter system, according to some embodiments;

FIG. 1D is a lower perspective view of the catheter hub slider of the catheter system, according to some embodiments;

FIG. 1E is a cross-sectional view of the catheter system in the first position, according to some embodiments;

FIG. 1F is a side view of a portion of the catheter system in the first position, according to some embodiments;

FIG. 1G is a top, partial cutaway view of the catheter system in the first position, according to some embodiments;

FIG. 2A is an upper perspective view of an example guidewire slider, according to some embodiments;

FIG. 2B is an upper perspective view of an example guidewire, according to some embodiments;

FIG. 2C is an upper perspective view of an example needle safety clip, according to some embodiments;

FIG. 2D is an upper perspective view of another example needle safety clip, according to some embodiments;

FIG. 3A is an upper perspective view of the catheter system in an example second position, according to some embodiments;

FIG. 3B is a lower perspective view of the catheter system in the second position, according to some embodiments;

FIG. 4A is an upper perspective view of the catheter system in an example third position, according to some embodiments;

FIG. 4B is an upper perspective view illustrating an example safety can coupled to the catheter hub slider in the third position, according to some embodiments;

FIG. 5A is an upper perspective view illustrating the safety can being uncoupled from the catheter hub slider as moved to an example fourth position, according to some embodiments;

FIG. 5B is an upper perspective view of the catheter system in the fourth position, according to some embodiments;

FIG. 6A is a lower perspective view of a portion of the catheter system in the fourth position, according to some embodiments;

FIG. 6B is an upper perspective view of the catheter system having an example introducer needle proximally withdrawn past the needle safety clip such that the needle safety clip is uncoupled from the catheter hub;

FIG. 7A is an upper perspective view of the catheter system having an example slot, the catheter system illustrated in the first position, according to some embodiments;

FIG. 7B is a cross-sectional view of the catheter system having the slot, the catheter system illustrated in the first position, according to some embodiments;

FIG. 7C is an upper perspective view of the catheter system having the slot, the catheter system illustrated in the second position, according to some embodiments;

FIG. 7D is an upper perspective view of the catheter system having the slot, the catheter system illustrated in the third position, according to some embodiments; and

FIG. 7E is an upper perspective view of the catheter system having the slot, the catheter system illustrated in the fourth position, according to some embodiments.

### DESCRIPTION OF EMBODIMENTS

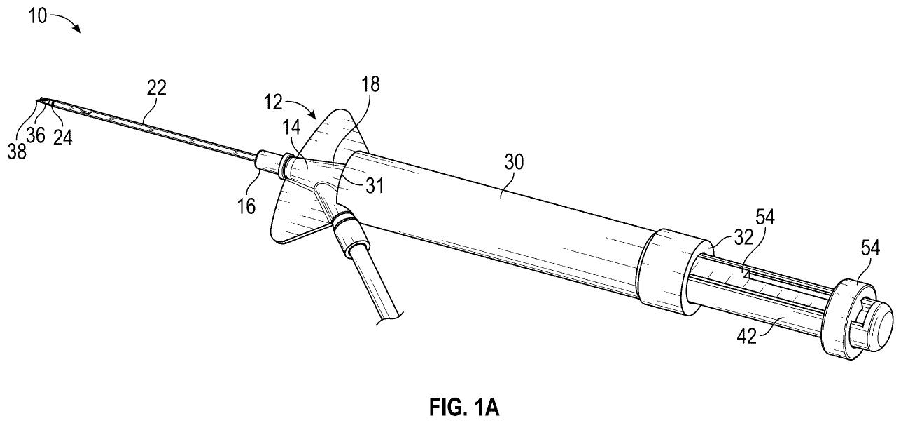

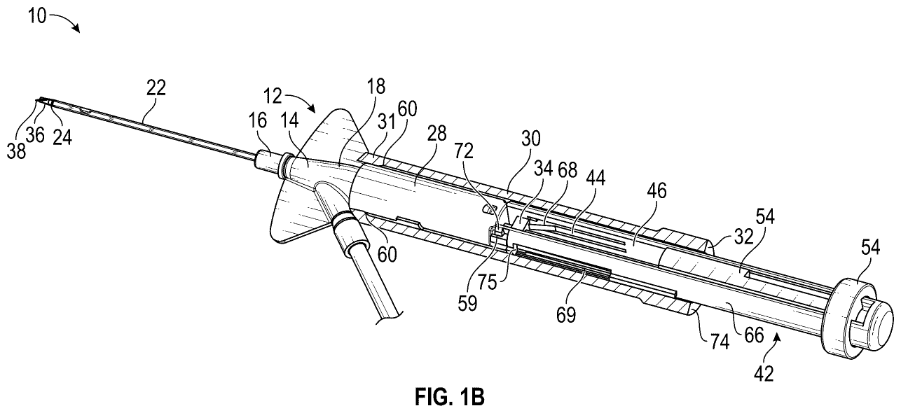

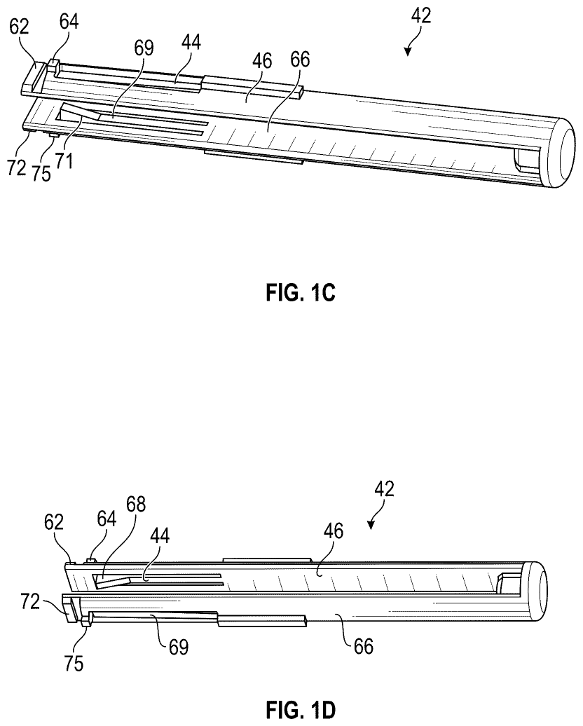

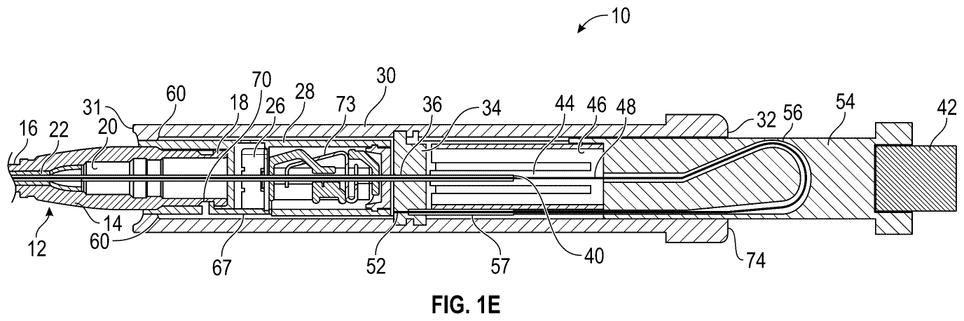

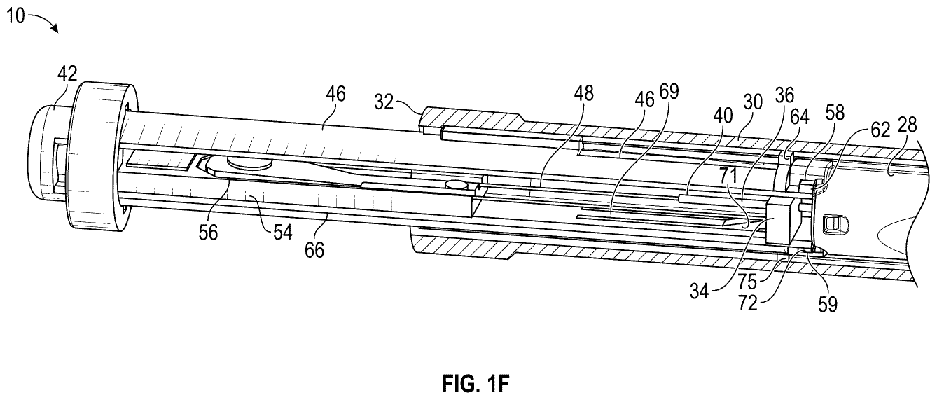

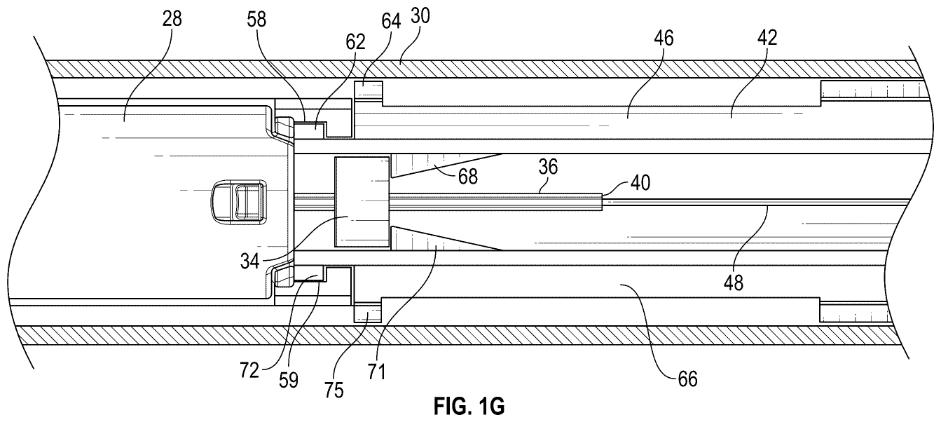

Referring now to FIGS. 1A-6B, in some embodiments, a catheter system 10 may include a catheter assembly 12, which may include a catheter hub 14. In some embodiments, the catheter hub 14 may include a distal end 16, a proximal end 18, and a lumen 20 extending through the distal end 16 of the catheter hub 14 and the proximal end 18 of the catheter hub 14. In some embodiments, the catheter system 10 may include a catheter 22, which may include a distal tip 24 and which may extend from the distal end 16 of the catheter hub 14. In some embodiments, the catheter system 10 may include a needle safety clip 26 disposed within a safety can 28 disposed proximal and/or proximate to the catheter hub 14.

In some embodiments, the catheter system 10 may include a barrel 30, which may include a distal end 31 and a proximal end 32. In some embodiments, the catheter system 10 may include a needle hub 34 fixed to the barrel 30. In some embodiments, the catheter system 10 may include an introducer needle 36, which may include a sharp distal tip 38 and a proximal end 40. In some embodiments, the proximal end 40 of the introducer needle 36 may be secured within the needle hub 34.

In some embodiments, the catheter system 10 may include a catheter hub slider 42, which may include a flexible arm 44. In some embodiments, the flexible arm 44 may be within and part of a distally-extending arm 46 of the catheter hub slider 42. In some embodiments, the catheter system 10 may include a guidewire 48, which may include a first end 50 and a second end 52. In some embodiments, the first end 50 of the guidewire 48 may be configured to advance distally through the introducer needle 36. In some embodiments, the catheter system 10 may include a guidewire slider 54.

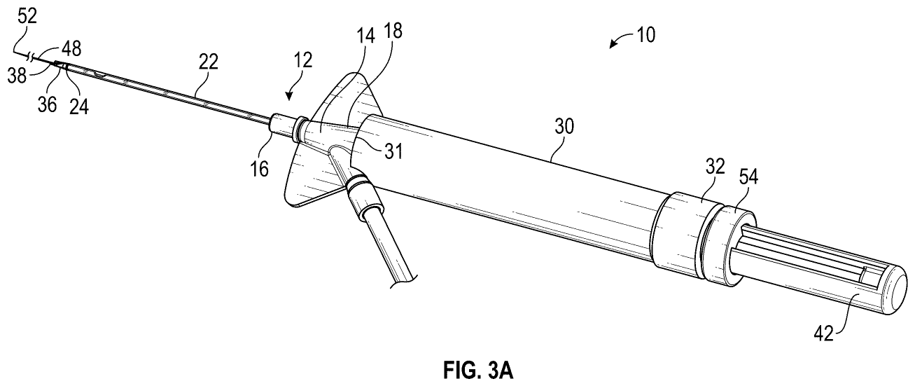

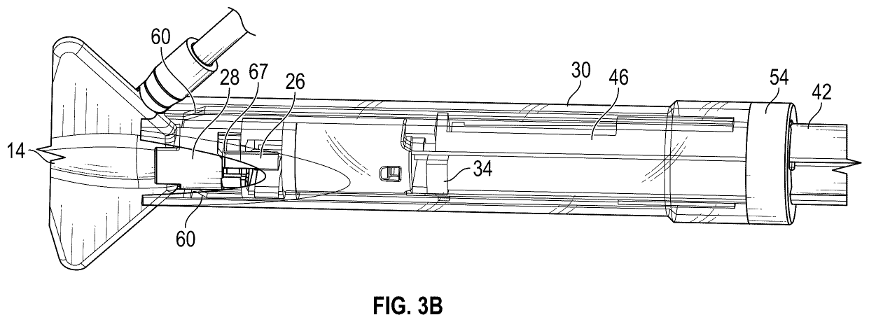

In some embodiments, the catheter system 10 may be configured to move from a first position, illustrated for example in FIGS. 1A-1B and 1E-1F, to a second position, illustrated for example in FIGS. 3A-3B. In some embodiments, when the catheter system 10 is in the first position, the guidewire slider 54 may be disposed in the guidewire slider proximal position and configured to slide from a guidewire slider proximal position to a distal position to distally advance the guidewire 48. In some embodiments, when the catheter system 10 is in the first position, the catheter hub slider 42 may be disposed in a catheter hub slider proximal position. In some embodiments, when the catheter system 10 is in the first position, the flexible arm 44 of the catheter hub slider 42 may contact the needle hub 34 to prevent the catheter hub slider 42 from sliding from a catheter hub slider proximal position to a distally-advanced position.

In some embodiments, when the catheter system 10 is in the second position, the guidewire slider 54 may be disposed in the distal position such that the guidewire 48 is advanced beyond the distal tip 24 of the catheter 22. In some embodiments, when the catheter system 10 is in the second position, the catheter hub slider 42 may be disposed in the catheter hub slider proximal position. In some embodiments, when the catheter system 10 is in the second position, the distally-extending arm 46 of the catheter hub slider 42 may be coupled to the catheter assembly 12 via the safety can 28 to prevent distal movement and proximal movement of the catheter hub slider 42 with respect to the catheter hub 14. In some embodiments, in response to the movement of the catheter system 10 from the first position to the second position, the guidewire slider 54 may push out on the flexible arm 44 such that the flexible arm 44 is removed from contact with the needle hub 34, and the catheter hub slider 42 is configured to slide from the catheter hub slider proximal position to the distally-advanced position. In some embodiments, when the catheter system 10 is in the second position, the guidewire slider 54 may be configured to slide back and forth between the guidewire slider proximal position and the distal position.

In some embodiments, the second end 52 of the guidewire 48 may be fixed within the barrel 30. In some embodiments, the second end 52 of the guidewire 48 may be fixed to the barrel 30. In some embodiments, the second end 52 of the guidewire 48 may be fixed within the needle hub 34. In some embodiments, the guidewire slider 54 may include a U-shaped slot 56 therethrough. In some embodiments, when the catheter system 10 is in the first position, the guidewire 48 may be disposed in the U-shaped slot 56 in a double-throw configuration such that there is a 2:1 advancement ratio between the first end 50 of the guidewire 48 and the guidewire slider 54. In some embodiments, the U-shaped slot 56 is oriented vertically within the barrel 30. In some embodiments, the catheter system 10 may include a support tubing 57, which may extend from the needle hub 34 and may facilitate support of the guidewire 48.

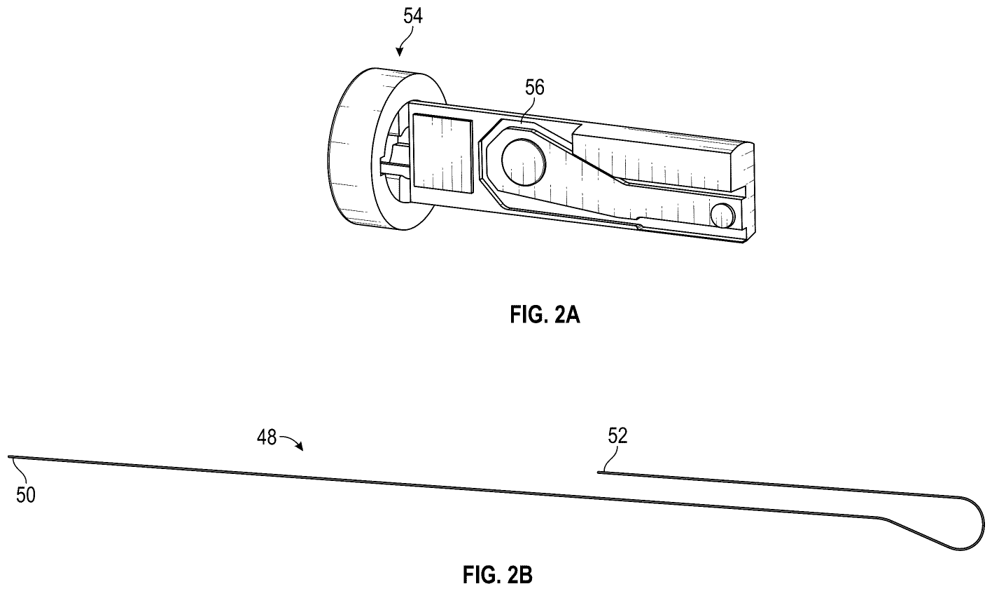

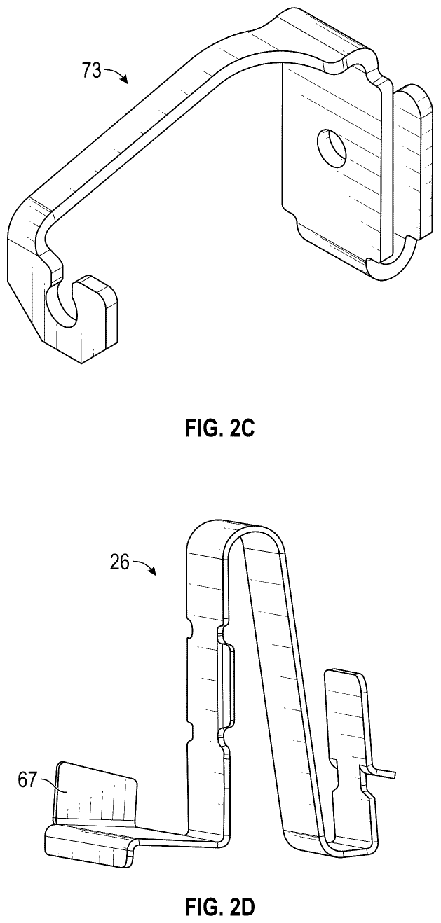

In some embodiments, the U-shaped slot 56 may be asymmetric. For example, as illustrated or example in FIG. 2A, the U-shaped slot 56 may be elongated towards a top of the catheter system 10, which may decrease an overall diameter or size of the barrel 30 for gripping by a clinician.

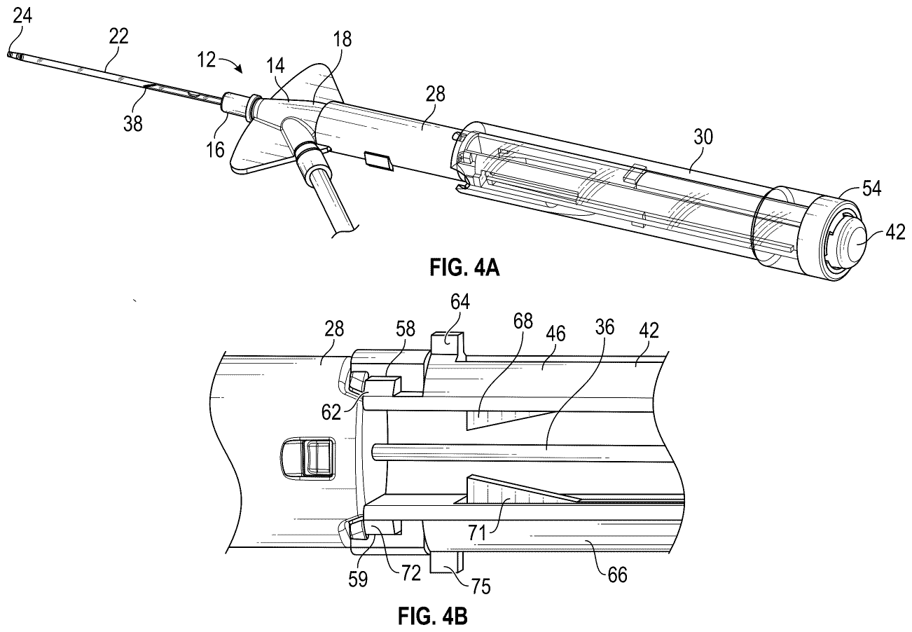

In some embodiments, the catheter 22 may be configured to move from the second position to a third position, illustrated for example in FIGS. 4A-4B. In some embodiments, when the catheter system 10 is in the third position, the guidewire slider 54 may be disposed in the distal position such that the guidewire 48 is advanced beyond the distal tip 24 of the catheter 22. In some embodiments, when the catheter system 10 is in the third position, the catheter hub slider 42 may be disposed in the distally-advanced position, and distal advancement of the catheter hub slider 42 advances the catheter assembly 12.

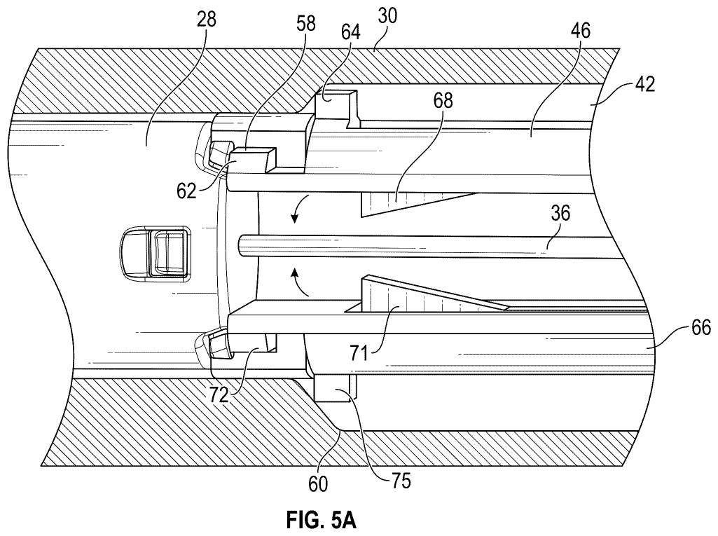

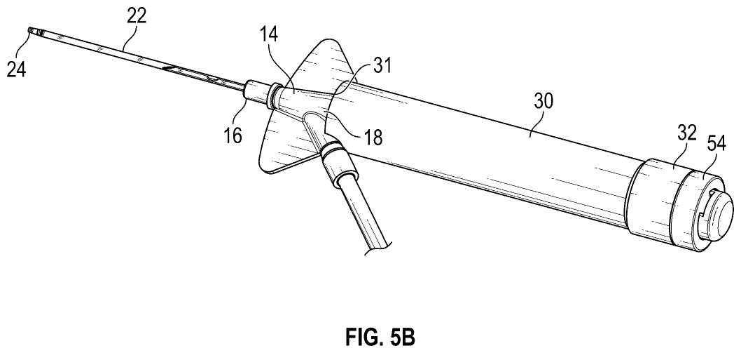

In some embodiments, the distally-advanced position of the catheter hub slider 42 is a partially distally-advanced position. In some embodiments, the safety can 28 may include one or more grooves 58. In some embodiments, the barrel 30 may include an inner ramp 60. In some embodiments, the catheter system 10 may be configured to move from the third position to a fourth position, illustrated for example in FIG. 5. In some embodiments, when the catheter system 10 is in the fourth position, the guidewire slider 54 may be disposed in the distal position such that the guidewire 48 is advanced beyond the distal tip 24 of the catheter 22. In some embodiments, when the catheter system 10 is in the fourth position, the catheter hub slider 42 may be disposed in a fully distally-advanced position.

In some embodiments, in response to movement of the catheter system 10 from the third position to the fourth position, the distally-extending arm 46 of the catheter hub slider 42 may contact the inner ramp 60 on the inner surface of the barrel 30 to remove the distally-extending arm 46 from the groove 58 of the safety can 28 such that the catheter hub slider 42 is uncoupled from the catheter assembly 12. In some embodiments, the distally-extending arm 46 may include a hook 62 and an outwardly-extending protrusion 64. In some embodiments, in response to movement of the catheter system 10 from the third position to the fourth position, the outwardly-extending protrusion 64 of the catheter hub slider 42 may contact the inner ramp 60 on the inner surface of the barrel 30 to remove the hook 62 from the groove of the safety can such that the catheter hub slider 42 is uncoupled from the catheter assembly 12.

In some embodiments, the distally-extending arm 46 may be a first distally-extending arm. In some embodiments, the catheter hub slider 42 may include a second distally-extending arm 66 opposite the first distally-extending arm. In some embodiments, in response to movement of the catheter system 10 from the third position to the fourth position, the second distally-extending arm 66 of the catheter hub slider 42 may contact the inner ramp 60 on the inner surface of the barrel 30 to remove the second distally-extending arm 66 from the groove 58 or another groove 59 of the safety can 28 such that the catheter hub slider 42 is uncoupled from the catheter assembly 12.

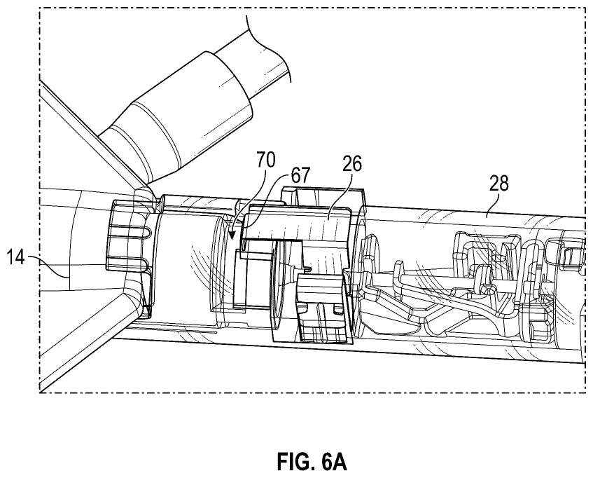

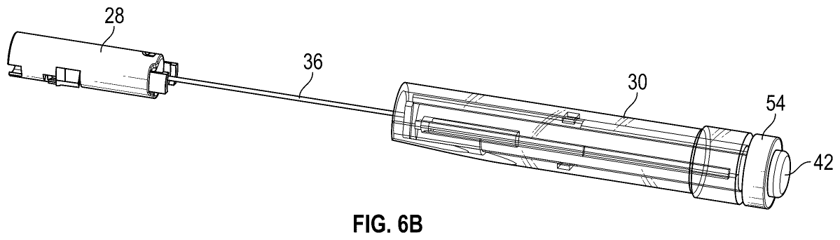

In some embodiments, when the catheter system 10 is in the fourth position, the barrel 30 may be moved proximally with respect to the catheter assembly 12 to withdraw the introducer needle 36 from the catheter assembly 12. In some embodiments, in response to proximal withdrawal of the introducer needle 36 past the needle safety clip 26, the needle safety clip 26 may uncouple from the catheter hub 14. As illustrated for example in FIG. 6A, in some embodiments, the needle safety clip 26 may include a V-shaped clip with a pawl 67 inserted into a groove 70 of the catheter hub 14. In some embodiments, in response to proximal withdrawal of the introducer needle 36 past the needle safety clip 26, the V-shaped clip may expand without compression by the introducer needle 36, causing release of the pawl 67 from the groove 70 and uncoupling from the catheter hub 14.

In some embodiments, when the catheter system 10 is in the first position, the second position, the third position, and the fourth position, the introducer needle 36 may extend through the catheter 22. As illustrated for example in FIG. 1B, in some embodiments, the flexible arm 44 may include a fin 68 tapered inwardly in a distal direction. As illustrated in FIG. 5A, in some embodiments, in response to the movement of the catheter system 10 from the first position to the second position, the guidewire slider may push out on the fin 68 such that the flexible arm 44 is removed from contact with the needle hub 34, and the catheter hub slider 42 is configured to slide from the catheter hub slider proximal position to the distally-advanced position.

In some embodiments, the flexible arm 44 may be a first flexible arm and/or the fin 68 may be a first fin. In some embodiments, the catheter hub slider 42 may include a second flexible arm 68 opposite the first flexible arm, as illustrated for example in FIGS. 1C-1D. In some embodiments, the second flexible arm 69 of the catheter hub slider 42, which may include a second fin 71, may contact the needle hub 34 to prevent the catheter hub slider 42 from sliding from the catheter hub slider proximal position to the distally-advanced position, as illustrated for example in FIG. 4B. In some embodiments, the hook 62 may be a first hook, and the outwardly-extending protrusion 64 may include a first outwardly-extending protrusion. In some embodiments, the second flexible arm 69 may include a second hook and/or a second outwardly-extending protrusion.

In some embodiments, in response to the movement of the catheter system 10 from the first position to the second position, the guidewire slider 54 may push out on the second flexible arm 69 such that the second flexible arm 69 is removed from contact with the needle hub 34 similar to the first flexible arm, and the catheter hub slider 42 is configured to slide from the catheter hub slider proximal position to the distally-advanced position. In some embodiments, the catheter system 10 may include another safety clip 73 disposed within the safety can 28 and configured to trap the sharp distal tip 38 of the introducer needle 36 in response to proximal withdrawal of the introducer needle 36.

In some embodiments, the proximal end 32 of the barrel 30 may include an opening 74. In some embodiments, the catheter hub slider 42 may be configured to slide within the opening 74 from the proximal catheter hub slider position to the distally-advanced position. In some embodiments, the guidewire slider 54 may be configured to slide within the opening 74 from the guidewire slider proximal position to the distal position. In some embodiments, the guidewire slider 54 may wrap around the catheter hub slider 42. In some embodiments, the guidewire slider 54 may wrap completely around the catheter hub slider 42. In some embodiments, a proximal end of the catheter hub slider 42 may include a vent plug configured to vent air, which may facilitate depression or distal movement of the catheter hub slider 42.

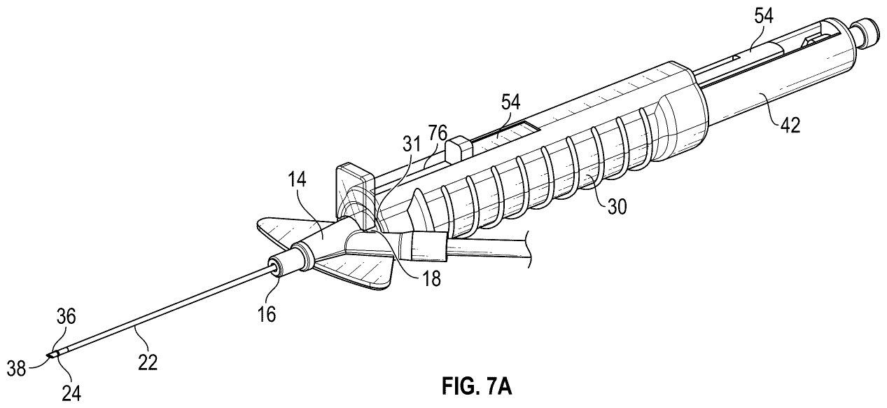

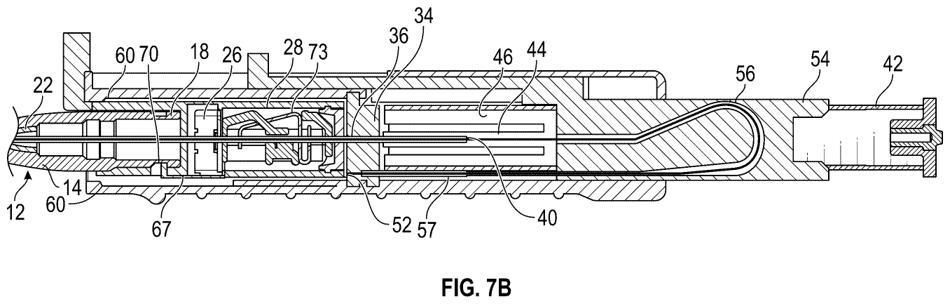

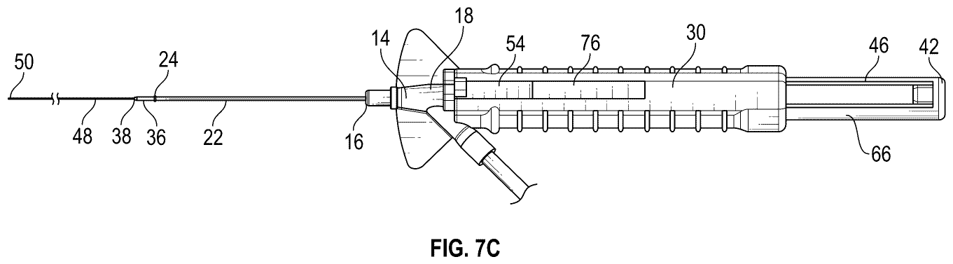

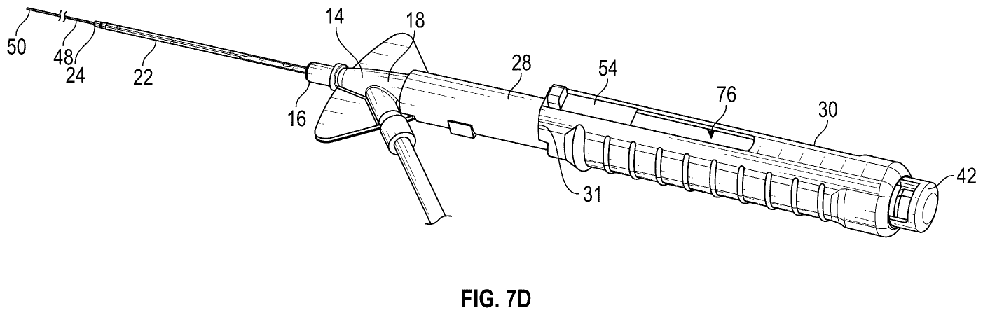

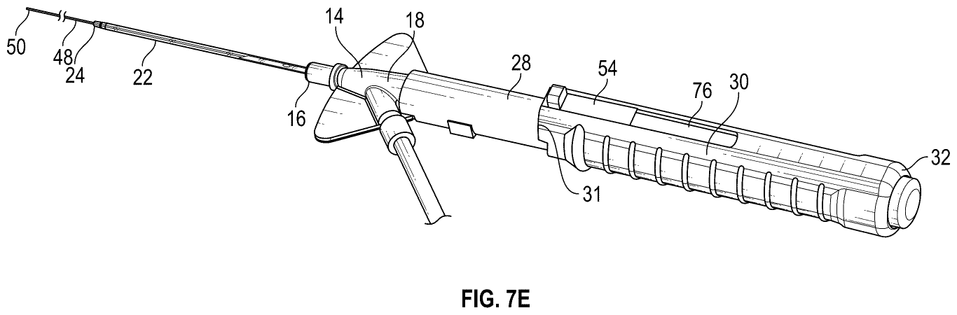

Referring now to FIGS. 7A-7E, the catheter system 10 is illustrated according to some embodiments. In some embodiments, the barrel 30 may include a slot 76 disposed between the distal end 31 of the barrel 30 and the proximal end 18 of the barrel 30. In some embodiments, when the catheter system 10 is in the first position, the guidewire slider 54 may extend through the slot 76 and may be configured to slide along the slot 76 from the guidewire slider proximal position to the distal position to distally advance the guidewire 48. In some embodiments, when the catheter system is in the second position, the guidewire slider 54 may extend through the slot 76 and may be configured to slide along the slot 76 from the guidewire slider distal position to the catheter hub proximal position. In some embodiments, an upper surface of the guidewire slider 54 may include an upwardly-extending protrusion 78, which may facilitate advancement and/or retraction of the guidewire slider 54 by a finger of the clinician. In some embodiments, the proximal end of the catheter hub slider 42 may include the vent plug configured to vent air, which may facilitate depression or distal movement of the catheter hub slider 42.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter system, comprising:
a catheter assembly, comprising:
a catheter hub, comprising a distal end, a proximal end, and a lumen extending through the distal end of the catheter hub and the proximal end of the catheter hub; and
a catheter comprising a distal tip and extending from the distal end of the catheter hub; and
a needle safety clip disposed within a safety can disposed proximal to the catheter hub;
a barrel, comprising a distal end and a proximal end;
a needle hub fixed to the barrel;
an introducer needle comprising a sharp distal tip and a proximal end, wherein the proximal end of the introducer needle is secured within the needle hub;
a catheter hub slider comprising a flexible arm within a distally-extending arm;
a guidewire comprising a first end and a second end, wherein the first end is configured to advance distally through the introducer needle;
a guidewire slider;
wherein the catheter system is configured to move from a first position to a second position, wherein when the catheter system is in the first position:
the guidewire slider is disposed in the guidewire slider proximal position and configured to slide from a guidewire slider proximal position to a distal position to distally advance the guidewire;
the catheter hub slider is disposed in a catheter hub slider proximal position;
the flexible arm of the catheter hub slider contacts the needle hub to prevent the catheter hub slider from sliding from a catheter hub slider proximal position to a distally-advanced position;
wherein when the catheter system is in the second position:
the guidewire slider is disposed in the distal position such that the guidewire is advanced beyond the distal tip of the catheter;
the catheter hub slider is disposed in the catheter hub slider proximal position;
the distally-extending arm of the catheter hub slider is coupled to the catheter assembly to prevent distal movement and proximal movement of the catheter hub slider with respect to the catheter hub;
wherein in response to the movement of the catheter system from the first position to the second position, the guidewire slider pushes out on the flexible arm such that the flexible arm is removed from contact with the needle hub, and the catheter hub slider is configured to slide from a catheter hub slider proximal position to a distally-advanced position.

2. The catheter system of claim 1, wherein the second end of the guidewire is fixed within the barrel, wherein the guidewire slider comprises a U-shaped slot therethrough, wherein when the catheter system is in the first position, the guidewire is disposed in the U-shaped slot in a double-throw configuration such that there is a 2:1 advancement ratio between the first end of the guidewire and the guidewire slider.

3. The catheter system of claim 2, wherein the U-shaped slot is oriented vertically within the barrel.

4. The catheter system of claim 3, wherein the U-shaped slot is asymmetric.

5. The catheter system of claim 1, wherein the catheter is configured to move from the second position to a third position, wherein when the catheter system is in the third position:
the guidewire slider is disposed in the distal position such that the guidewire is advanced beyond the distal tip of the catheter;
the catheter hub slider is disposed in the distally-advanced position, wherein distal advancement of the catheter hub slider advances the catheter assembly.

6. The catheter system of claim 5, wherein the distally-advanced position of the catheter hub slider is a partially distally-advanced position, wherein the safety can comprises a groove, wherein the barrel further comprises an inner ramp, wherein the catheter system is configured to move from the third position to a fourth position, wherein when the catheter system is in the fourth position:
the guidewire slider is disposed in the distal position such that the guidewire is advanced beyond the distal tip of the catheter;
the catheter hub slider is disposed in a fully distally-advanced position;
wherein in response to movement of the catheter system from the third position to the fourth position, the distally-extending arm of the catheter hub slider contacts the inner ramp on an inner surface of the barrel to remove the distally-extending arm from the groove of the safety can such that the catheter hub slider is uncoupled from the catheter assembly.

7. The catheter system of claim 6, wherein the distally-extending arm comprises a hook and an outwardly-extending protrusion, wherein in response to movement of the catheter system from the third position to the fourth position, the outwardly-extending protrusion of the catheter hub slider contacts the inner ramp on the inner surface of the barrel to remove the hook from the groove of the safety can such that the catheter hub slider is uncoupled from the catheter assembly.

8. The catheter system of claim 7, wherein the distally-extending arm is a first distally-extending arm, wherein the catheter hub slider further comprises a second distally-extending arm opposite the first distally-extending arm, wherein in response to movement of the catheter system from the third position to the fourth position, the second distally-extending arm of the catheter hub slider contacts the inner ramp on the inner surface of the barrel to remove the second distally-extending arm from the groove of the safety can such that the catheter hub slider is uncoupled from the catheter assembly.

9. The catheter system of claim 6, wherein when the catheter system is in the fourth position, the barrel may be moved proximally with respect to the catheter assembly to withdraw the introducer needle from the catheter assembly, wherein in response to proximal withdrawal of the introducer needle past the needle safety clip, the needle safety clip uncouples from the catheter hub.

10. The catheter system of claim 9, wherein when the catheter system is in the first position, the second position, the third position, and the fourth position, the introducer needle extends through the catheter.

11. The catheter system of claim 1, wherein the flexible arm comprises a fin tapered inwardly in a distal direction, wherein in response to the movement of the catheter system from the first position to the second position, the guidewire slider pushes out on the fin such that the flexible arm is removed from contact with the needle hub, and the catheter hub slider is configured to slide from the catheter hub slider proximal position to the distally-advanced position.

12. The catheter system of claim 1, wherein the flexible arm is a first flexible arm, wherein the catheter hub slider comprises a second flexible arm opposite the first flexible arm, wherein the second flexible arm of the catheter hub slider contacts the needle hub to prevent the catheter hub slider from sliding from the catheter hub slider proximal position to the distally-advanced position, wherein in response to the movement of the catheter system from the first position to the second position, the guidewire slider pushes out on the second flexible arm such that the flexible arm is removed from contact with the needle hub, and the catheter hub slider is configured to slide from a catheter hub slider proximal position to a distally-advanced position.

13. The catheter system of claim 1, wherein the catheter assembly further comprises another safety clip disposed within the safety can and configured to trap the sharp distal tip of the introducer needle in response to proximal withdrawal of the introducer needle.

14. The catheter system of claim 1, wherein the barrel further comprises a slot disposed between the distal end of the barrel and the proximal end of the barrel, wherein when the catheter system is in the first position, the guidewire slider extends through the slot and is configured to slide along the slot from the guidewire slider proximal position to the distal position to distally advance the guidewire.

15. The catheter system of claim 14, wherein when the catheter system is in the second position, the guidewire slider extends through the slot and is configured to slide along the slot from the guidewire slider distal position to the catheter hub proximal position.

16. The catheter system of claim 1, wherein the proximal end of the barrel comprises an opening, wherein the catheter hub slider is configured to slide within the opening from the proximal catheter hub slider position to the distally-advanced position, wherein the guidewire slider is configured to slide within the opening from the guidewire slider proximal position to the distal position.

17. The catheter system of claim 16, wherein the guidewire slider wraps around the catheter hub slider.

18. The catheter system of claim 17, wherein the guidewire slider wraps completely around the catheter hub slider.

19. The catheter system of claim 1, wherein the second end of the guidewire is fixed within the needle hub.

20. The catheter system of claim 1, wherein when the catheter system is in the second position, the guidewire slider is configured to slide back and forth between the guidewire slider proximal position and the distal position.
